(19) United States
(12) Patent Application Publication
(10) Pub. No.: US 2013/0164346 A1
(43) Pub. Date: Jun. 27, 2013

(54) METHOD FOR PREPARING BIOMEDICAL METAL ALLOY MATERIAL WITH MULTI-DRUG DELIVERY SYSTEM

(71) Applicant: Kwungpook National University Industry Academic Cooperation Foundation, Daegu (KR)

(72) Inventors: Kyu Bok Lee, Daegu (KR); Jun Sik Son, Daegu (KR); Tae Yub Kwon, Daegu (KR); Kyo Han Kim, Daegu (KR)

(73) Assignee: Kwungpook National University Industry Academic Cooperation Foundation, Daegu (KR)

(21) Appl. No.: 13/718,169

(22) Filed: Dec. 18, 2012

(30) Foreign Application Priority Data
Dec. 22, 2011 (KR) 10-2011-01400179

(51) Int. Cl. A61K 47/02; A61K 9/00

(52) U.S. Cl. CPC A61K 47/02; A61K 9/00
USPC 424/400; 427/2.24

(57) ABSTRACT

The present invention provides a method for preparing a biomedical metal alloy material with a multi-drug delivery system. A biomedical metal alloy material with a multi-drug delivery system according to the present invention is prepared by incorporating a therapeutic agent into a biodegradable material to prepare particles containing the therapeutic agent, treating the surface of the particles containing the therapeutic agent to have a charge opposite to the surface charge of a metal alloy material, and inducing an electrostatic interaction between the surface charges of the particles containing the therapeutic agent and the metal alloy material to immobilize the surface-treated particles containing the therapeutic agent on the surface of the metal alloy material.

### CROSS-REFERENCE TO RELATED APPLICATION

This application claims priority to and the benefit of Korean Patent Application No. 10-2011-140179, filed on Dec. 22, 2011, the disclosure of which is incorporated herein by reference in its entirety.

Acknowledgment: This work was supported by a grant from Korea Healthcare Technology R&D Project, Ministry of Health, Welfare and Family Affairs, Republic of Korea (A091074).

### BACKGROUND

1. Field of the Invention

The present invention relates to a method for preparing a biomedical metal alloy material with a multi-drug delivery system.

2. Discussion of Related Art

Metal alloys for biomedical implants are biomaterials with excellent physical properties, mechanical properties, and biocompatibilities such as strength, fatigue resistance, moldability, corrosion resistant etc., compared to other materials such as ceramics, polymers, etc., and thus are most widely used in dental, orthopedic, and plastic surgeries for artificial hip joints, stents, etc. and for the purpose of regeneration and treatment of defects and damaged areas of bone as a hard tissue. Examples of the metal alloys include iron, chrome, nickel, stainless steel, cobalt-based alloys, titanium, titanium alloys, zirconium, niobium, tantalum, gold, silver, etc., and among them, stainless steel, titanium, titanium alloys, gold, etc. that have excellent corrosion resistance and exhibit stable characteristics in human tissues, compared to other metal materials, are most widely used in the human body. When these metal alloys are applied to the human body, two conditions should be satisfied. First, it should have an excellent biocompatibility. That is, when the metal alloys are used as devices for body support, they should not cause foreign body reactions and toxicity to surrounding tissues. Moreover, when the metal alloys are used as bone replacement implants (such as missing tooth, missing bones etc) and bone formation supports (such as scaffold, bone graft substitutes etc), they should have bone conduction mechanisms to promote bone ingrowth and osteoinductive elements to induce bone regeneration and restoration by recruiting and stimulating mesenchymal stem cells of the host. Second, the metal alloys should have surface functions and structural integrity such that osteogenic cells differentiated at various stages during bone formation and fracture healing and newly formed bone can fuse perfectly with the surface of implanted metal alloys. The metal alloys with these functions can be used as substitutes for ideal bone regeneration and healing. However, most of the currently available metal alloy materials do not have these properties (osseointegration, osteoconduction, osteoinduction). Accordingly, in order to improve the disadvantages of the above-described metal alloy materials, many trials have been conducted over the past few years to increase the surface area of metal alloys, to change the surface topograph, and to improve bone adhesion by physical and chemical surface treatments. However, no effective method has been developed so far.

In general, the surface of the metal material plays an important role in diverse cell activities such as cell adhesion, proliferation, differentiation, apoptosis, etc. as well as protein adsorption by interaction with cells. Similarly, metal ions and therapeutic agents (drugs, proteins, peptides, DNA, RNA, etc) released from the metal material affect the activities of cells. Accordingly, the surface properties of the metal alloys are the most important properties as a starting point causing a series of reactions between the material surface, adhesion molecules, and cell membrane receptors. Thus, since the early 1990s, numerous attempts have been made in the surface modification with the aims of enhancing osseointegration, minimizing absorption of bone around metal alloy implants, and improving compatibility with and adhesion to surrounding soft tissues. Examples of typical surface treatment methods of metal ally materials include sintering of metal beads (Amigo et al, J. Mat. Proc. Technol., 141, 117-122 (2003)), blasting and acid treatment (Felghan et al, J. Bone and Joint Surg., 77, 1380-1395 (1995)), alkali immersion and heat treatment (Kim et al, J. Mater. Sci. Mater. Med., 8, 341-347 (1997)), hydroxyapatite coating (Popa et al, J. Mater. Sci. Mater. Med., 16, 1165-1171 (2005)), anodic oxidation (Yang al, J. Biomaterials, 25, 1003-1010 (2004)), ion implantation (Rautray et al, J. Biomed. Mater. Res. B Appl. Biomater., 93, 581-591 (2010)), etc. The control of the surface design has been steadily studied, and it has been reported that the clinical effects of surface-treated metal alloy implants are better than those of non-surface-treated metal alloy implants.

As such, the biomedical metal alloy implants are currently studied to fuse perfectly with newly formed tissues by physicochemical surface treatment. However, the surface of the metal alloy implant has no function of inducing and regenerating hard tissues such as bone, etc. and soft tissues such as cartilage, etc., and thus direct and rapid bone regeneration will not be possible. Therefore, technologies for coating therapeutic agents, which are effective in tissue induction, on the surface of metal alloys have been studied.

Methods for coating therapeutic agents on the surface of implants can be generally divided into three categories. The first is to coat a functional polymer mixed with a therapeutic agent on an implant surface. This method for coating the functional polymer has problems that the polymer is easily degraded and that the biocompatibility is low. The second is to form a hydroxyapatite coating layer on the implant surface and then to physically adsorb a therapeutic agent thereon. This physical adsorption method has a problem that it is difficult to control the release rate of the therapeutic agent adsorbed on the surface. The third is to simultaneously mix hydroxyapatite with a therapeutic agent by biomimetic coating, in which hydroxyapatite crystals formed by precipitating Ca²⁺ and PO₄³⁻ ions from an aqueous solution containing Ca and P at an appropriate pH are coated on the implant surface. The addition of the therapeutic agent to the aqueous solution allows the hydroxyapatite and the therapeutic agent to be simultaneously coated on the implant surface. However, since the biomimetic method uses the precipitation of ions, the deposition rate of the coating layer is very low, typically less than 0.5 μm per hour, the coating process is complicated, the accurate control of the concentration of the therapeutic agent in the coating layer is difficult, the addition of the therapeutic agent at high concentration is difficult, and the adhesion between the coating layer and the surface of the metal material is low, resulting in limitations in industrial applications.

Techniques related to the methods for coating the therapeutic agent on the implant surface include a method for coating a polymer material mixed with an antibiotic on the surface of the metal implant (Diefenbeck et al, Injury, Int. J. Care Injured (2006) 37, S95-S104). Moreover, Korean Patent No. 10-1070345 discloses an organic/inorganic hybrid coating layer containing a bioactive factor, a biomedical implant, and a manufacturing method thereof, Korean Patent Application Publication No. 10-2007-42245 discloses a composition containing biodegradable polymers, growth factors and aqueous solvent for implants coating, and a coating method using electrospray, and Korean Patent No. 10-892866 discloses a method for immobilizing therapeutic agents on an implant surface.

As mentioned above, with the development of various materials, the physical functions of biocompatible metal alloy materials have obtained remarkable progress over the past several decades, but many biological functions for tissue regeneration and wound healing are lacking. In particular, with the recent research on tissue engineering using a variety of stem cells present in the human body (such as adipose stem cells, umbilical cord stem cells, amniotic stem cells, bone marrow stem cells, mesenchymal stem cells, etc), methods for effectively achieving tissue regeneration and wound healing by using correlation between metal alloy materials and stem cells, i.e., by inducing proliferation and differentiation of stem cells in metal alloy materials have been studied, and thus the physiological functions of the metal alloy materials have attracted significant attention.

Meanwhile, biopolymers (including natural and synthetic polymers) are widely used as supports for tissue regeneration and carriers for drug delivery. In particular, according to the biopolymers as drug carriers, hydrophilic and hydrophobic therapeutic agents can be effectively encapsulated in polymer particles by various methods, and further the size of polymer particles can be controlled from several tens of nanometers to several hundreds of micrometers by physical processes. These biopolymer particles has an advantage that the release of the encapsulated drug can be controlled from several weeks to several months depending on the molecular weight and composition of the polymer used, the size and porosity of the particles, etc.

However, the methods for coating biopolymers containing therapeutic agents on the surface of metal alloys, which have been studied until now, have problems that the process is somewhat complicated, the biocompatibility is low, the adhesion between the coating layer and the surface of the metal material is low, and the drug release is difficult to control.

Thus, the need to develop a biomedical metal alloy material, which can be prepared by a simple process, has excellent biocompatibility, can improve adhesion between a therapeutic agent coating layer and the surface of a metal material, and can effectively control drug release, is urgently required.

### SUMMARY OF THE INVENTION

The inventors of the present invention have studied a biomedical metal alloy material, which has excellent biocompatibility, can improve adhesion between a therapeutic agent coating layer and the surface of a metal material, and can effectively control drug release, and found that it is possible to effectively control the number, content, and release rate of therapeutic agents to be introduced into a metal alloy material by incorporating the therapeutic agent into a biodegradable material to prepare particles containing the therapeutic agent, treating the surface of the particles containing the therapeutic agent to have a charge opposite to the surface charge of the metal alloy material, and inducing an electrostatic interaction between the surface charges of the particles containing the therapeutic agent and the metal alloy material to immobilize the surface-treated particles containing the therapeutic agent on the surface of the metal alloy material, thereby completing the present invention.

Thus, an object of the present invention is to provide a method for preparing a biomedical metal alloy material with a multi-drug delivery system.

In one aspect, the present invention provides a method for preparing a biomedical metal alloy material with a multi-drug delivery system, comprising the steps of:

(1) incorporating a therapeutic agent into a biodegradable material to prepare particles containing the therapeutic agent;

(2) coating the surface of the particles containing the therapeutic agent with an ionic polymer having a positive charge or a negative charge to treat the surface of the particles containing the therapeutic agent to have a charge opposite to the surface charge of a metal alloy material; and

(3) dispersing the surface-treated particles containing the therapeutic agent in an aqueous solution, adding the metal alloy material thereto, and stirring the resulting mixture to induce an electrostatic interaction between the surface charges of the particles containing the therapeutic agent and the metal alloy material to immobilize the particles containing the therapeutic agent on the surface of the metal alloy material.

Hereinafter, the present invention will be described in detail.

A biomedical metal alloy material with a multi-drug delivery system according to the present invention is prepared by incorporating a therapeutic agent into a biodegradable material to prepare particles containing the therapeutic agent, treating the surface of the particles containing the therapeutic agent to have a charge opposite to the surface charge of a metal alloy material, and inducing an electrostatic interaction between the surface charges of the particles containing the therapeutic agent and the metal alloy material to immobilize the surface-treated particles containing the therapeutic agent on the surface of the metal alloy material.

The method for preparing the biomedical metal alloy material with a multi-drug delivery system according to the present invention will be described in detail step by step below.

Step (1) is to prepare particles containing a therapeutic agent by incorporating the therapeutic agent into a biodegradable material.

The biodegradable material is not particularly limited and may include any material that can be used for the purpose of biomedical applications. For example, the biodegradable material may comprise at least one selected from the group consisting of polydioxanone, polyglycolic acid, polylactic acid, polycaprolactone, lactic acid-glycolic acid copolymer, glycolic acid-trimethylcarbonate, glycolic acid-ε-caprolactone, polyglyconate, polyglactin, polyamino acid, polyanhydride, polyorthoester, and mixtures and copolymers thereof; collagen, gelatine, chitin/chitosan, alginate, albumin, hyaluronic acid, heparin, fibrinogen, cellulose, dextran, pectin, polylysine, polyethyleneimine, dexamethasone, chondroitin sulfate, lysozyme, DNA, RNA, protein derivatives such as Arg-Gly-Asp (RGD), etc.; and lipid, growth factors, growth hormones, peptide drugs, protein drugs, anti-inflammatory analgesic agents, anticancer agents, antiviral agents, sex hormones, antibiotics, antimicrobial agents, and mixtures thereof. Preferably, the biodegradable material may comprise at least one selected from the group consisting of polydioxanone, polyglycolic acid, polylactic acid, lactic acid-glycolic acid copolymer, and mixtures thereof, which are approved as biodegradable materials that can be used in the human body by the U.S. Food and Drug Administration (FDA); collagen, gelatine, chitin/chitosan, alginate, albumin, hyaluronic acid, heparin, fibrinogen, cellulose, dextran, pectin, chondroitin sulfate, lysozyme, DNA, RNA, Arg-Gly-Asp (RGD), etc.; and growth factors, growth hormones, sex hormones, and mixtures thereof.

The therapeutic agent may comprise at least one selected from the group consisting of growth factors, growth hormones, peptide drugs, protein drugs, anti-inflammatory analgesic agents, anticancer agents, antiviral agents, sex hormones, antibiotics, antimicrobial agents, and mixtures thereof. For example, the therapeutic agent may comprise at least one selected from the group consisting of grow factors such as transforming growth factors, fibroblast growth factors, bone morphogenetic proteins, vascular endothelial growth factors, epidermal growth factors, insulin-like growth factors, platelet-derived growth factors, nerve growth factors, hepatocyte growth factors, placental growth factors, granulocyte colony-stimulating factors, etc.; growth hormones such as animal growth hormones, human growth hormones, etc.; peptide or protein drugs such as chondroitin sulfate, heparin, erythropoietin, granulocyte colony-stimulating factors, interferon, follicle-stimulating hormones, luteinizing hormones, goserelin acetate, leuprorelin acetate, decapeptyl, luteinizing hormone-releasing hormone agonists, etc.; anti-inflammatory analgesic agents such as dexamethasone, indomethacin, ibuprofen, ketoprofen, piroxicam, flurbiprofen, diclofenac, etc.; anticancer agents such as paclitaxel, doxorubicin, camptothecin, 5-fluorouracil, cytosine arabinose, methotrexate, etc.; antiviral agents such as acyclovir, robavin, tamiflu, etc.; sex hormones such as testosterone, estrogen, progesterone, estradiol, etc.; antibiotics such as tetracycline, minocycline, doxycycline, ofloxacin, levofloxacin, ciprofloxacin, clarithromycin, erythromycin, cefaclor, cefotaxime, imipenem, penicillin, gentamicin, streptomycin, vancomycin, etc.; antifungal agents such as ketoconazole, itraconazole, fluconazole, amphotericin-B, nystatin, griseofulvin, etc.; and compounds such as β-glycerophosphate, ascorbate, hydrocortisone, 5-azacytidine, etc., but not limited thereto.

The method for preparing the particles containing the therapeutic agent is not particularly limited and may include any method that is used in the art to incorporate the therapeutic agent into the biodegradable material. Preferably, a water-in-oil emulsion method, a water-in-oil-in-water emulsion method, a spraying method, a solvent diffusion method, a phase separation method, a method for forming an intermolecular electrolyte complex between materials with charges, a liposome method, etc. may be used. More preferably, the emulsion method or the electrolyte complex method may be used.

The particles containing the therapeutic agent may be in a form in which the therapeutic agent is incorporated into the biodegradable material by the emulsion method or in the form of a complex by the electrolyte complex method. Moreover, the particles containing the therapeutic agent may independently contain at least two therapeutic agents.

The therapeutic agent contained in the particles may be present in an amount suitable for an implantation target or implantation site of the biomedical metal alloy material and/or for treatment or prevention of related diseases. The amount of the therapeutic agent can be controlled based on various factors such as the implantation target's age, sex, general health state, and weight, the type and severity of a disease, the process of manufacturing the metal alloy material, the type and content of the biodegradable material used in the preparation of the particles, the dosage and administration period of the therapeutic agent, etc. and the amount suitable for each factor may be appropriately selected by a person having ordinary skill in the art.

The particles containing the therapeutic agent may be prepared in the form of spheres or powder. Moreover, the particles containing the therapeutic agent may be non-porous or may be prepared to have a porosity of 5% to 98%, and preferably 5% to 90%. The porous particles may be formed of various pore-forming materials well known in the art. Preferably, within the range of porosity, it is possible to effectively control the release rate of the therapeutic agent of the particles. For example, in the case of a therapeutic agent that requires a fast release rate, the release rate can be increased with a higher porosity.

Moreover, the particles containing the therapeutic agent may have a diameter from several tens of nanometers to several hundreds of micrometers, and preferably 10 nm to 500 μm. If the diameter of the particles containing the therapeutic agent exceeds 500 μm, it is difficult to immobilize the particles on the surface of the metal alloy material. Moreover, if the metal alloy material has a porous structure, the porosity of the material may be reduced, which is not desirable. Furthermore, if the diameter of the particles containing the therapeutic agent is less than 10 nm, the particles cannot contain a sufficient amount of therapeutic agent, which is also not desirable.

Step (2) is to treat the surface of the particles containing the therapeutic agent to have a charge opposite to the surface charge of the metal alloy material. First, in order to provide hydrophilicity to the surface of the particles containing the therapeutic agent, the surface of the particles is modified by plasma treatment. Then, the surface of the particles containing the therapeutic agent, which is hydrophilically modified, is coated with an ionic polymer having a positive charge or a negative charge to have a charge opposite to the surface charge of the metal alloy material.

The plasma treatment is to allow particles having high energy to collide with the surface of a certain material such that the energy is transferred to the surface of the material and may be performed in a non-vacuum environment or in a vacuum of less than 200 mtorr in the presence of a gas at 10 to 200 Watt for 1 to 5 minutes. As the gas used in the plasma treatment, oxygen, argon, hydrogen peroxide, or ammonia gas may be used, and preferably, oxygen or argon gas may be used.

The ionic polymer having a positive charge or a negative charge may comprise at least one cationic polymer selected from the group consisting of polyethyleneimine, polylysine, polyallylamine, polyvinylamine, poly(diallyldimethylammonium chloride), poly methyl aminoethyl methacrylate, N-hydroxysuccinimide, N-3-dimethylaminopropyl-N′-ethylcarbodiimide hydrochloride, chitosan, lysozyme, dextran, protein, and vancomycin; or at least one anionic polymer selected from the group consisting of polydioxanone, polyglycolic acid, polylactic acid, polycaprolactone, lactic acid-glycolic acid copolymer, glycolic acid-trimethylcarbonate, glycolic acid-ε-caprolactone, polyglyconate, polyglactin, and copolymers thereof, collagen, heparin, albumin, hyaluronic acid, chondroitin sulfate, hydrochloride carboxymethylcellulose, sodium tripolyphosphate, polystyrene sulfonate, gelatin, and alginate, but not limited thereto.

Step (3) is to induce an electrostatic interaction between the surface charges of the particles containing the therapeutic agent and the metal alloy material to immobilize the particles containing the therapeutic agent on the surface of the metal alloy material. First, in order to activate the surface charge of the metal alloy material, the surface is modified. Then, the particles containing the therapeutic agent surface-treated in step (2) are dispersed in an aqueous solution, the surface-modified metal alloy material is added thereto, and the resulting mixture is gently shake, thus immobilizing the particles containing the therapeutic agent on the surface of the metal alloy material.

The metal alloy material may include any material that can be used for the purpose of biomedical applications and include artificial synthetic metal alloys and natural metal alloys. Preferably, the metal alloy material may include iron, chrome, nickel, stainless steel, cobalt-based alloy, titanium, titanium alloy, zirconium, niobium, tantalum, gold, and silver. Moreover, the metal alloy material may include at least one selected from the group consisting of stainless steel, titanium, titanium alloy, and gold, which have excellent corrosion resistance and exhibit stable characteristics in human tissues, compared to other metal materials. Furthermore, the metal alloy material may have any form and, for example, may have at least one form selected from the group consisting of block, film, filament, fiber, membrane, mesh, woven fabric/non-woven fabric, knit, grains, particles, plate, bolt/nut, and nail. Preferably, the form of a block with a rough surface is more advantageous for tissue regeneration. The metal alloy material may be completely non-porous or may have a porosity of 5 to 98% and a pore size of 0.1 nm to 5 mm.

The surface modification of the metal alloy material may be performed by at least one method selected from the group consisting of plasma treatment, sintering of metal beads, blasting and acid treatment, alkali immersion and heat treatment, ceramic coating, anodic oxidation, ion implantation, and combinations thereof.

The plasma treatment may be performed in the same conditions as the plasma treatment described in step (2). By the plasma treatment, it is possible to further increase the electrostatic interaction between the particles containing the therapeutic agent and the metal alloy material through the hydrophilic surface modification of the particles containing the therapeutic agent and/or the activation of the surface charge of the metal alloy material.

Moreover, the ceramic coating may use at least one material selected from the group consisting of calcium phosphate-based ceramics such as hydroxyapatite (HA), tricalcium phosphate (TCP), tetracalcium phosphate (TTCP), dicalcium phosphate anhydrous (DCPA), etc.; bioactive glasses such as silica-based glasses, phosphate-based glasses, glass ceramics, etc; alumina; zirconia; and complexes thereof. The bioactive glass refers to any glass that exhibits bioactive properties and is an amorphous solid material capable of providing adhesive bonding to both hard and soft tissues, although it is not adhesive, when it is exposed to an appropriate in vivo environment and a laboratory environment such as a simulated body fluid (SBF) or trishydroxymethylaminomethane buffer solution.

The ceramic coating on the surface of the metal alloy material may be performed by at least one method selected from the group consisting of ion beam sputtering, radio-frequency sputtering, pulsed laser deposition, plasma spray, super high speed blast coating, and simulated body fluid method, but not limited thereto.

The process of immobilizing the particles containing the therapeutic agent on the surface of the metal alloy material may be performed by dispersing the particles containing the therapeutic agent in an appropriate solvent and immersing the metal alloy material in the resulting solution without any separate treatment as long as the particles containing the therapeutic agent and the metal alloy material have opposite surface charges. The solvent in which the particles are dispersed is not particularly limited and may include any solvent that does not have a significant effect on the surface charge characteristics of the particles or the metal alloy material. Preferably, the solvent may include at least one selected from the group consisting of water, ethanol, methanol, acetone, heptane, pentane, and mixtures thereof at a pH of 2 to 9.

When the surface charges of the particles containing the therapeutic agent and the metal alloy material are the same, the electrostatic interaction may be induced by coating the surface of the particles containing the therapeutic agent or the metal alloy material with a material having a charge opposite to the surface charge of the particles containing the therapeutic agent or the metal alloy material. For example, if the particles containing the therapeutic agent have a negative surface charge and the metal alloy material also has a negative surface charge, the surface of the particles containing the therapeutic agent or the metal alloy material having the negative charge is coated with a material having a positive charge to have an opposite charge to induce the electrostatic interaction, thereby immobilizing the particles on the surface of the metal alloy material.

The immobilized particles containing the therapeutic agent may be in an amount of 10⁻⁷ wt % to 90 wt %, and preferably 10⁻⁵ wt % to 50 wt % with respect to the total weight of the metal alloy material. If the amount of the immobilized particles containing the therapeutic agent exceeds 90 wt %, the particles are excessively immobilized on the surface of the metal alloy material, and thus the excellent surface function of the metal alloy material may be inhibited, which is not desirable. On the contrary, if the amount of the immobilized particles is less than 10⁻⁷ wt %, only a small amount of therapeutic agent can be incorporated into the metal alloy material, which is also not desirable.

As a post-treatment step to improve the adhesion between the surface of the metal alloy material and the particles containing the therapeutic agent immobilized thereon or to improve the bioactivity of the surface of the metal alloy material, the present invention may further comprise the step of physically treating the metal alloy material on which the particles are immobilized. That is, the method of the present invention may further comprise the step of performing the post-treatment by at least one step selected from the group consisting of (a) immersing the metal alloy material, on which the particles containing the therapeutic agent are immobilized, in a solvent and drying the resulting metal alloy material, (b) partially melting the metal alloy material on which the particles containing the therapeutic agent are immobilized, and (c) coating the surface of the metal alloy material, on which the particles containing the therapeutic agent are immobilized, with apatite. When the post-treatment step is performed, it is possible to prevent the particles, immobilized on the surface of the metal alloy material, from being separated and to prevent the therapeutic agent from being lost after the metal alloy material is applied in the body, thus further improving the effect of the therapeutic agent.

In step (a) of immersing the metal alloy material, on which the particles containing the therapeutic agent are immobilized, in a solvent and drying the resulting metal alloy material, the metal alloy material, on which the particles containing the therapeutic agent are immobilized, may be immersed in at least one solvent selected from the group consisting of water, hydrochloric acid, acetic acid, ethanol, acetone, methanol, dichloromethane, chloroform, toluene, acetonitrile, 1,4-dioxane, tetrahydrofuran, hexafluoroisopropanol, and mixtures thereof and dried. The time required for the immersing process may vary depend on the type of the biodegradable material used in the formation of the particles and may preferably be selected from a range that does not deteriorate the properties of the therapeutic agent contained in the particles or the metal alloy material.

Step (b) of partially melting the metal alloy material on which the particles containing the therapeutic agent are immobilized may be performed when the particles containing the therapeutic agent comprise a thermoplastic polymer or when the therapeutic agent contained in the particles is susceptible to an organic solvent. The melting treatment may be performed by dry or wet heat, and the range of the heat and the time of the melting treatment may be appropriately controlled depending on the type of the biodegradable material used. Preferably, the melting treatment may be performed at 30° C. to 300° C. for 10 seconds to 1 hour, more preferably at 30° C. to 150° C. for 10 seconds to 10 minutes, and most preferably at 50° C. to 100° C. for 30 seconds to 5 minutes.

Step (c) of coating the surface of the metal alloy material, on which the particles containing the therapeutic agent are immobilized, with apatite may be performed to allow the apatite to be more readily formed on the surface of the metal alloy material, on which the particles containing the therapeutic agent are immobilized, by an alternating immersion process of (i) immersing the metal alloy material, on which the particles containing the therapeutic agent are immobilized, in a 30% (v/v) to 90% (v/v) ethanol aqueous solution, in which 0.1 M to 1 M calcium chloride (CaCl₂) is dissolved, for 3 to 10 seconds, immersing the resulting metal alloy material in a pure 30% (v/v) to 90% (v/v) ethanol aqueous solution for 1 to 3 seconds, and drying the resulting metal alloy material at room temperature for 3 to 5 minutes and then (ii) immersing the resulting metal alloy material in a 30% (v/v) to 90% (v/v) ethanol aqueous solution, in which 0.1 M to 1 M dipotassium phosphate (K₂HPO₄) is dissolved, for 3 to 10 seconds, immersing the resulting metal alloy material in a pure 30% (v/v) to 90% (v/v) ethanol aqueous solution for 1 to 3 seconds, and drying the resulting metal alloy material at room temperature for 3 to 5 minutes, the alternating immersion process being performed one to five times. When the alternating immersion process is performed in the above manner, it is possible to form the apatite on the surface of the particles, immobilized on the surface of the metal alloy material, and the surface of the metal alloy material within several days and to effectively improve the bioactivity of the surface of the metal alloy material for hard tissue regeneration.

Then, the metal alloy material, on which the particles containing the therapeutic agent are immobilized, subjected to the alternating immersion process may be immersed in a simulated body fluid (SBF) solution. The SBF solution may have a concentration of 1 to 5 times, and the immersion process may be performed at a temperature of 37±0.5° C. at a pH of 6.4 to 7.4 for 1 hour to 5 days, and preferably for 1 hour to 2 days.

When the surface of the metal alloy material is coated with the apatite, it is possible to lower the release rate of the therapeutic agent contained in the particles and to prevent the particles containing the therapeutic agent, immobilized on the surface of the metal alloy material, from being separated, thus further improving the delivering effect of the therapeutic agent.

The metal alloy material, on which the particles containing the therapeutic agent are immobilized, prepared by the above method may be applied to the human body as any metal alloy material that can be used in direct contact with living biological tissues for the purpose of biological regeneration, biological replacement, support or treatment. For example, the metal alloy material according to the present invention can be applied to supports, joints, bone fixation devices, spine fixation devices, etc. for hard tissue regeneration such as artificial bones, artificial joints, bone cements, jaw bones, maxillofacial bones, heart valves, blood vessels, dental and orthopedic implants, abutments, fillers, porcelains, brackets, cores, posts, etc. Moreover, the metal alloy material according to the present invention can be used for other application-related drug deliveries, vascular contrast agents, micro-electro-mechanical systems (MEMSs), antimicrobial fillers, hybrid complexes, etc.

Thus, the biomedical metal alloy material according to the present invention can be used for the purpose of regeneration of hard tissue and soft tissues, healing of wounds, treatment of diseases, etc. and can be prepared using any therapeutic agent having a charge, regardless of the raw material, shape, strength, porosity, surface function, etc. of the designed metal alloy material. Moreover, particles containing a plurality of therapeutic agent of different types can be simply and effectively incorporated on the surface of the metal alloy material. Furthermore, the number, content, and release rate of therapeutic agents to be introduced into the metal alloy material can be effectively controlled, and thus it is possible to prepare a biomedical metal alloy material with a multi-drug delivery system which significantly improves the cell affinity to implantation sites, inflammatory reaction, tissue regeneration, and healing effect and to effectively use the biomedical metal alloy material in any implantable biological device that can actively regenerate tissues.

### BRIEF DESCRIPTION OF THE DRAWINGS

The above and other objects, features and advantages of the present invention will become more apparent to those of ordinary skill in the art by describing in detail exemplary embodiments thereof with reference to the accompanying drawings, in which:

FIG. 1 shows scanning electron microscopy (SEM) images of the surface morphologies of sand-blasted titanium, on which PLGA particles containing vancomycin are immobilized, prepared in Example 1 of the present invention (A: before post-treatment and B: after post-treatment);

FIG. 2 shows SEM images of the surface morphologies of hydroxyapatite (HA)-coated titanium, on which PLGA particles containing vancomycin and dexamethasone are immobilized, prepared in Example 2 of the present invention (A: before post-treatment and B: after post-treatment);

FIG. 3 shows SEM images of the surface morphologies of titanium, on which PLGA particles containing rhBMP-2 are immobilized, prepared in Example 4 of the present invention (A: before post-treatment and B: after post-treatment); and

FIG. 4 shows a change in release behavior of a therapeutic agent before and after apatite coating titanium, on which PLGA particles containing rhBMP-2 are immobilized, with apatite prepared in Example 4 of the present invention.

### DETAILED DESCRIPTION OF EXEMPLARY EMBODIMENTS

Hereinafter, preferred examples will be presented for a better understanding of the present invention. It is to be understood, however, that the following examples are given for illustrative purpose only and are not construed to limit the scope of the present invention.

### Example 1

### Preparation of Titanium Implant Immobilized with PLGA Particles Loaded with Vancomycin

1-1. Preparation of Titanium Discs with Rough Surface

Titanium discs with a diameter of 5 mm and a height of 3 mm were prepared by casting. Then, the surface of the titanium discs was sand-blasted to form a rough structure thereon.

1-2. Preparation of PLGA Particles Loaded with Vancomycin

PLGA particles loaded with (or containing) vancomycin were prepared by a double emulsion method. First, 1 g of poly(lactic-co-glycolic acid) (PLGA) (monomer ratio 75:25) was completely dissolved in 10 ml of dichloromethane, a solution in which 40 mg of vancomycin, a bioactive drug, was dissolved in 1 ml of water was added thereto, and the resulting mixture was emulsified using a homogenizer at 20,000 rpm for 3 minutes to prepare a suspension. The prepared suspension was added to 100 ml of 0.2 wt % polyvinylalcohol (PVA) aqueous solution and emulsified using a homogenizer at 20,000 rpm for 3 minutes. Then, the mixture was poured into 200 ml of 0.5 wt % PVA aqueous solution and stirred at 600 rpm for 5 hours. Subsequently, the mixture was washed with distilled water three times and freeze-dried to prepare PLGA particles containing vancomycin.

It was observed that the average diameter of the prepared PLGA particles loaded with vancomycin was 2 μm, the amount of vancomycin contained in the PLGA particles was 920 ng, and the loading efficiency of vancomycin was 41%.

1-3. Surface Treatment of PLGA Particles Loaded with Vancomycin

The PLGA particles loaded with vancomycin prepared in Example 1-2 were subjected to hydrophilic surface modification with oxygen plasma treatment at 30 Watt for 3 minutes. The hydrophilic surface-modified PLGA particles loaded with vancomycin were dispersed in 0.05 wt % polyethyleneimine (PEI) aqueous solution and stirred for 12 hours to coat the surface of the PLGA particles loaded with vancomycin with a positively charged material. Then, the reaction mixture was centrifuged in a centrifuge at 3,000 rpm for 5 minutes to collect the PEI-coated PLGA particles, and the collected PEI-coated PLGA particles were washed with distilled water three times to remove PEI and then freeze-dried at −20° C.

1-4. Immobilization of PLGA Particles Loaded with Vancomycin on Titanium Surface

The sand-blasted titanium discs prepared in Example 1-1 were subjected to surface modification with oxygen plasma at 30 Watt for 3 minutes to activate the surface of the titanium discs. Then, 5 mg of vancomycin-loaded PLGA particles coated with PEI, a positively charged polymer, prepared in Example 1-3 were dispersed in distilled water. Ten titanium discs surface-modified with the plasma treatment were added to the distilled water in which the PEI-coated PLGA particles loaded with vancomycin were dispersed, and slowly stirred for 4 hours. Then, the resulting titanium discs were washed with distilled water at least three times to remove an excess of PLGA particles, which were not immobilized on the surface, and completely dried at room temperature. As a result, the PLGA particles containing vancomycin coated with PEI, a positively charged polymer, were immobilized on the titanium surface with a negatively charged surface.

It was observed that the average amount of the PLGA particles containing vancomycin immobilized on the titanium surface was 2.3 μg.

1-5. Post-Treatment Step

In order to improve the adhesion between the PLGA particles containing vancomycin and the titanium surface, the titanium, on which the PLGA particles containing vancomycin were immobilized, prepared in Example 1-4 was immersed in a 80% (v/v) ethanol aqueous solution for 3 minutes and dried at room temperature, thus preparing a titanium implant with a drug delivery system on which the PLGA particles containing vancomycin were immobilized.

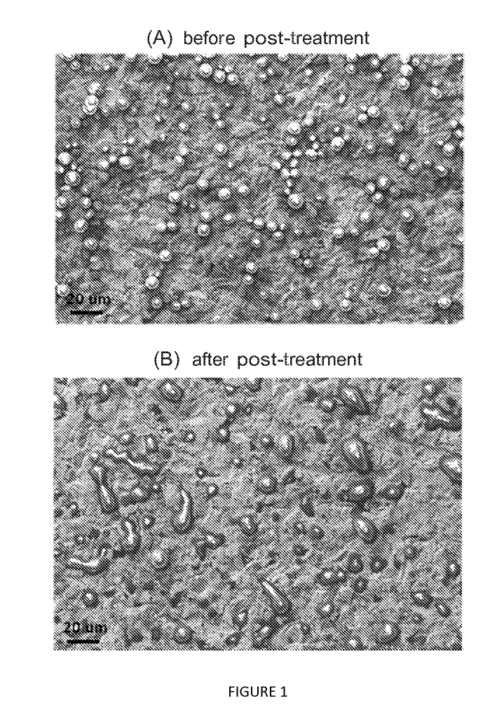

The surface morphologies of the sand-blasted titanium on which the PLGA particles containing vancomycin were immobilized were observed with a scanning electron microscope (SEM) and shown in FIG. 1 (A: before post-treatment and B: after post-treatment).

As shown in FIG. 1, it was found that the PEI-coated PLGA particles containing vancomycin were well dispersed and immobilized on the titanium surface. Moreover, it was found that the PLGA particles containing vancomycin after the post-treatment were completely immobilized on the titanium surface, compared to the PLGA particles before the post-treatment. As such, the PLGA particles can be completed immobilized on the titanium surface through the post-treatment, and thus it is possible to effectively prevent the PLGA particles present on the titanium surface from being separated when the titanium material is implanted into the human body.

### Example 2

### Preparation of Hydroxyapatite (HA)-Coated Titanium Implant on which PLGA Particles Double-Containing Vancomycin and Dexamethasone (DEX) are Immobilized

2-1. Preparation of HA-Coated Titanium Discs

Titanium discs with a diameter of 5 mm and a height of 3 mm were prepared by casting. Then, the surface of the titanium discs was coated with hydroxyapatite (HA) nanoparticles using a low temperature high speed collision (LTHSC) method.

2-2. Preparation of PLGA Particles Containing Vancomycin and PLGA Particles Containing Dexamethasone (DEX)

PLGA particles containing vancomycin were prepared by a water-in-oil emulsion method. First, 500 mg of PLGA (monomer ratio 75:25) was completely dissolved in 8 ml of dichloromethane, a solution in which 30 mg of vancomycin, a bioactive drug, was dissolved in 3 ml of DMSO was added thereto, and the resulting mixture was emulsified using a homogenizer at 20,000 rpm for 3 minutes to prepare a suspension. The prepared suspension was added to 100 ml of 0.1 wt % polyvinylalcohol (PVA) aqueous solution and stirred at 500 rpm for 5 hours. Then, the reaction mixture was centrifuged in a centrifuge at 3,000 rpm for 5 minutes to collect PLGA particles, and the collected PLGA particles were washed with distilled water three times and then freeze-dried at −20° C., thus preparing the PLGA particles containing vancomycin.

PLGA particles containing dexamethasone were prepared in the following manner. In detail, 1 g of PLGA (monomer ratio 50:50) was completely dissolved in 9 ml of dichloromethane, a solution in which 40 mg of dexamethasone, a bioactive drug, was dissolved in 1 ml of acetone was added thereto, and the resulting mixture was stirred for 20 minutes to prepare a suspension. The prepared suspension was added to 100 ml of 0.2 wt % polyvinylalcohol (PVA) aqueous solution and emulsified using a homogenizer at 10,000 rpm for 3 minutes, and the resulting mixture was poured into 300 ml of 0.5 wt % PVA aqueous solution and stirred at 600 rpm for 5 hours. Then, the reaction mixture was centrifuged in a centrifuge at 3,000 rpm for 5 minutes to collect PLGA particles, and the collected PLGA particles were washed with distilled water three times and then freeze-dried at −20° C., thus preparing the PLGA particles containing dexamethasone.

It was observed that the average diameter of the prepared PLGA particles containing vancomycin was 800 nm, the amount of vancomycin contained in the PLGA particles was 672 ng, and the loading efficiency of vancomycin was 56%.

Moreover, it was observed that the average diameter of the prepared PLGA particles containing dexamethasone was 2 μm, the amount of dexamethasone contained in the PLGA particles was 510 ng, and the loading efficiency of dexamethasone was 34%.

2-3. Surface Treatment of PLGA Particles Containing Vancomycin and Dexamethasone

The PLGA particles containing vancomycin and the PLGA particles containing dexamethasone prepared in Example 2-2 were coated with a positively charged PEI in the same manner as Example 1-3.

2-4. Immobilization of PLGA Particles Containing Vancomycin and Dexamethasone on HA Film-Coated Titanium Surface

The PLGA particles containing vancomycin and the PLGA particles containing dexamethasone, which were coated with PEI, a positively charged polymer, prepared in Example 2-3 were mixed in a weight ratio of 1:1 and dispersed in distilled water. Then, the titanium discs were placed in the distilled water in which the PLGA particles were dispersed, and slowly stirred for 4 hours. Subsequently, the resulting titanium discs were washed with distilled water at least three times to remove an excess of PLGA particles, which were not immobilized on the surface, and completely dried at room temperature.

It was observed that the average amount of the PLGA particles containing vancomycin immobilized on the HA-coated titanium surface was 1.2 μg and the average amount of the PLGA particles containing dexamethasone immobilized on the HA film-coated titanium surface was 1.5 μg.

2-5. Post-Treatment Step

In order to improve the adhesion between the PLGA particles containing vancomycin and dexamethasone and the HA-coated titanium surface, the HA-coated titanium, on which the PLGA particles containing vancomycin and dexamethasone were immobilized, prepared in Example 2-4 was treated in a chamber maintained at 80° C. for 3 minutes to prepare an HA-coated titanium implant with two types of drug delivery systems, in which the PLGA particles containing vancomycin and dexamethasone were immobilized on the surface.

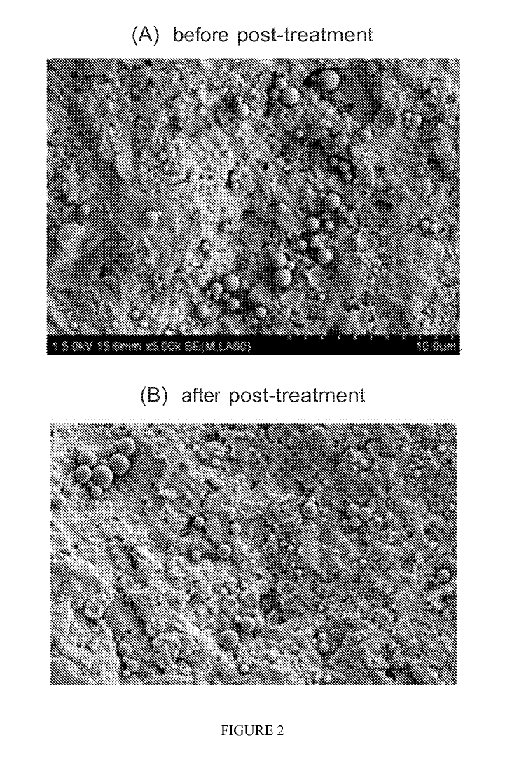

The surface morphologies of the HA-coated titanium on which the PLGA particles containing vancomycin and dexamethasone were immobilized were observed with a scanning electron microscope (SEM) and shown in FIG. 2 (A: before post-treatment and B: after post-treatment).

As shown in FIG. 2, it was found that the PEI-coated PLGA particles containing vancomycin and dexamethasone were well dispersed and immobilized on the HA-coated titanium surface. Moreover, it was found that the PLGA particles after the post-treatment were more firmly immobilized on the HA-coated titanium surface than the PLGA particles before the post-treatment.

### Example 3

### Preparation of Titanium Implant on which PLGA Particles Triple-Containing rhBMP-2/Heparin, Vancomycin, and Dexamethasone are Immobilized

3-1. Preparation of Titanium Discs with Rough Surface

Titanium discs with a diameter of 5 mm and a height of 3 mm were prepared by casting. Then, the surface of the titanium discs was sand-blasted to form a rough structure thereon.

3-2. Preparation of PLGA Particles Triple-Containing rhBMP-2/Heparin, Vancomycin, and Dexamethasone

PLGA particles containing rhBMP-2/heparin were prepared in the following manner. First, 40 mg of PLGA (monomer ratio 75:25) was completely dissolved in 2 ml of DMSO, a 5% (w/v) Pluronic F-127 aqueous solution in which heparin (Aldrich Co.) was dissolved was slowly added dropwise thereto, thus preparing heparin-functionalized PLGA particles. The heparin-functionalized PLGA particles were collected by centrifugation at 12,000 rpm for 1 hour, and 8 mg of PLGA particles were dispersed again in 40 μl of phosphate buffer solution (PBS). Then, 4 mg of rhBMP-2 (R&D System Co.) was added to the PBS in which the PLGA particles were dispersed and slowly stirred at 4° C. for 6 hours. Subsequently, a suspension of PLGA particles on which rhBMP-2 was immobilized by an electrostatic interaction with heparin was placed in a dialysis tube, and the resulting dialysis tube was immersed in 15 ml of PBS and gently stirred at 37° C. for 6 hours to remove unreacted rhBMP-2, thus preparing PLGA particles containing rhBMP-2/heparin.

PLGA particles containing vancomycin and PLGA particles containing dexamethasone were prepared in the same manners as in Example 1-2 and Example 2-2.

It was observed that the average size of the prepared PLGA particles containing rhBMP-2/heparin was 800 nm, the amount of rhBMP-2 contained in the PLGA particles was 720 ng, and the loading efficiency of rhBMP-2 was 90%.

Moreover, it was observed that the average size of the prepared PLGA particles containing vancomycin was 800 nm, the amount of vancomycin contained in the PLGA particles was 672 ng, and the loading efficiency of vancomycin was 56%.

Furthermore, it was observed that the average size of the prepared PLGA particles containing dexamethasone was 2 μm, the amount of dexamethasone contained in the PLGA particles was 510 ng, and the loading efficiency of dexamethasone was 34%.

3-3. Surface Treatment of PLGA Particles Triple-Containing rhBMP-2/Heparin, Vancomycin, and Dexamethasone

The PLGA particles containing vancomycin and the PLGA particles containing dexamethasone, except for the PLGA particles containing rhBMP-2/heparin among the PLGA particles containing rhBMP-2/heparin, the PLGA particles containing vancomycin, and the PLGA particles containing dexamethasone, prepared in Example 3-2, were coated with a positively charged PEI in the same manner as in Example 1-3.

3-4. Immobilization of PLGA Particles Triple-Containing rhBMP-2/Heparin, Vancomycin, and Dexamethasone on Titanium Surface

The PLGA particles containing vancomycin, the PLGA particles containing dexamethasone, and the PLGA particles containing rhBMP-2/heparin, which were coated with PEI, a positively charged polymer, prepared in Example 3-3 were mixed in a weight ratio of 1:1:1 and dispersed in distilled water. Then, the titanium discs prepared in Example 3-1 were placed in the distilled water in which the PLGA particles were dispersed, and slowly stirred for 4 hours. Subsequently, the resulting titanium discs were washed with distilled water at least three times to remove an excess of PLGA particles, which were not immobilized on the surface, and completely dried at room temperature.

It was observed that the average amount of the PLGA particles containing rhBMP-2/heparin immobilized on the titanium surface was 0.8 μg, and it was found that the prepared PLGA particles triple-containing rhBMP-2/heparin, vancomycin, and dexamethasone were well dispersed and immobilized on the titanium surface (not shown).

### Example 4

### Preparation of Titanium Implant on which PLGA Particles Containing rhBMP-2 are Immobilized

4-1. Preparation of Titanium Discs

Titanium discs with a diameter of 5 mm and a height of 3 mm were prepared by casting and were not sand-blasted.

4-2. Preparation of PLGA Particles Containing rhBMP-2

PLGA particles containing rhBMP-2 were prepared by a water-in-oil-in-water double emulsion method in the following manner. First, 300 mg of PLGA (monomer ratio 50:50) was dissolved in 5 ml of dichloromethane, a solution in which 10 μg of rhBMP-2 (R&D System Co) was dissolved in 1 ml of 4 mM HCl was added thereto, and the resulting mixture was emulsified using a homogenizer at 12,000 rpm for 3 minutes. Then, the mixture was poured into a 0.5% (w/v) PVA aqueous solution and stirred at 500 rpm for 6 hours. Subsequently, the mixture was centrifuged in a centrifuge at 5,000 rpm for 10 minutes to collect PLGA particles, and the collected PLGA particles were washed with distilled water three times and then freeze-dried at −20° C., thus preparing the PLGA particles containing rhBMP-2.

It was observed that the average diameter of the prepared PLGA particles containing rhBMP-2 was 5 μm, the amount of rhBMP-2 contained in the PLGA particles was 288 ng, and the loading efficiency of rhBMP-2 was 12%.

4-3. Surface Treatment of PLGA Particles Containing rhBMP-2

The PLGA particles containing rhBMP-2 prepared in Example 4-2 were coated with a positively charged PEI in the same manner as Example 1-3.

4-4 Immobilization of PLGA Particles Containing rhBMP-2 on Titanium Surface

5 mg of the PLGA particles containing rhBMP-2 coated with PEI, a positively charged polymer, prepared in Example 4-3 were dispersed in distilled water. Then, the titanium discs prepared in Example 4-1 were placed in the distilled water in which the PLGA particles were dispersed, and slowly stirred for 4 hours. Subsequently, the resulting titanium discs were washed with distilled water at least three times to remove an excess of PLGA particles, which were not immobilized on the surface, and freeze-dried at −20° C., thus preparing the titanium on which the PLGA particles containing rhBMP-2 were immobilized. It was observed that the average amount of the PLGA particles containing rhBMP-2 immobilized on the titanium surface was 2.4 μg.

4-5. Post-Treatment Step by Apatite Coating

A 1-time simulated body fluid (SBF) solution was prepared by adding 8.035 g of NaCl, 0.355 g of NaHCO₃, 0.225 g of KCl, 0.231 g of K₂HPO₄.3H₂O, 0.311 g of MgCl₂.6H₂O, 0.292 g of CaCl₂, 0.072 g of Na₂SO₄, 6.118 g of (HOCH₂)₃(CNH₂), and 39 ml of 1.0M HCl to 1 L of distilled water. Then, in order to provide a precursor that can form apatite on the titanium disc surface on which the PLGA particles containing rhBMP-2 were immobilized, an alternating immersion process of immersing the titanium disc, on which the PLGA particles containing rhBMP-2 were immobilized, prepared in Example 4-4 in a 30% ethanol aqueous solution, in which 100 mM calcium chloride was dissolved, for 10 seconds, immersing the resulting titanium disc in a pure 30% (v/v) to 90% (v/v) ethanol aqueous solution for 1 second, drying the resulting titanium disc at room temperature for 3 minutes, immersing the resulting titanium disc in a 30% ethanol aqueous solution, in which 100 mM dipotassium phosphate was dissolved, for 10 seconds, immersing the resulting titanium disc in a pure 30% ethanol aqueous solution for 1 second, and drying the resulting titanium disc at room temperature for 3 minutes, was repeated three times. The resulting titanium disc was immersed in the prepared SBF solution having a concentration of 1-time at 37° C. for 24 hours, thus preparing apatite-coated titanium disc with a long-term drug delivery system.

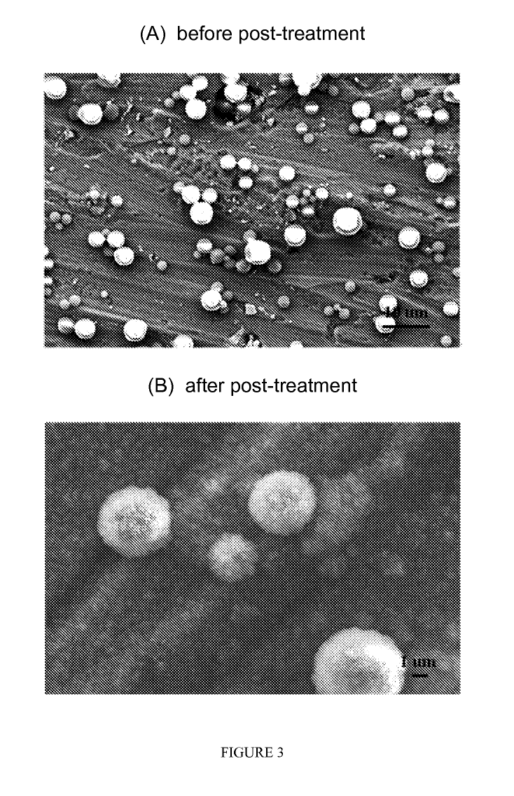

The surface morphologies of the titanium on which the PLGA particles containing rhBMP-2 were immobilized were observed with a scanning electron microscope (SEM) and shown in FIG. 3 (A: before post-treatment and B: after post-treatment).

As shown in FIG. 3, it was found that the PEI-coated PLGA particles containing rhBMP-2 were well dispersed and immobilized on the titanium surface. Moreover, it was found that the PLGA particles after the post-treatment were more firmly immobilized on the titanium surface than the PLGA particles before the post-treatment.

### Example 5

### Preparation of Titanium Implant on which Chondroitin Sulfate Particles Containing rhBMP-2 and Bovine Serum Albumin (BSA) Particles Containing Vancomycin are Immobilized

5-1. Preparation of Titanium Discs with Rough Surface

Titanium discs with a diameter of 5 mm and a height of 3 mm were prepared by casting. Then, the surface of the titanium discs was sand-blasted to form a rough structure thereon.

5-2. Preparation of Chondroitin Sulfate Particles Containing rhBMP-2 and BSA Particles Containing Vancomycin

Chondroitin sulfate (CS) particles containing rhBMP-2 were prepared using ionic complexes thereof. First, an rhBMP-2 aqueous solution was prepared by dissolving 10 μg of rhBMP-2 in 500 μl of PBS, and a chondroitin sulfate aqueous solution (pH 7.4) was prepared by dissolving 10 μg of chondroitin sulfate in 500 μl of PBS. The rhBMP-2 aqueous solution was added to the chondroitin sulfate aqueous solution and stirred for 1 hour to allow the rhBMP-2 having a positive charge and the chondroitin sulfate having a negative charge to form particles by their opposite charges, thus preparing the chondroitin sulfate particles containing rhBMP-2.

BSA particles containing vancomycin were prepared by a nanoprecipitation method. First, a BSA aqueous solution was prepared by dissolving 100 mg of BSA in 10 ml of PBS, and a vancomycin aqueous solution was prepared by dissolving 50 mg of vancomycin in 10 ml of PBS. 1 ml of 10 mM NaCl aqueous solution was added to 1 ml of the BSA aqueous solution and gently stirred, and 100 μl of the vancomycin aqueous solution was added thereto and left at 4° C. for 1 hour. Then, the reaction solution was stirred with a stirrer at 700 rpm, and 2 ml of acetone was added thereto and stirred for 1 hour, thus preparing the BSA particles containing vancomycin.

It was observed that the average diameter of the prepared chondroitin sulfate particles containing rhBMP-2 was 80 nm, the amount of rhBMP-2 contained in the chondroitin sulfate particles was 1.7 μg, and the loading efficiency of rhBMP-2 was 92%. Moreover, it was observed that the average diameter of the prepared BSA particles containing vancomycin was 250 nm, the amount of vancomycin contained in the BSA particles was 2.6 μg, and the loading efficiency of vancomycin was 95%.

5-3. Surface Treatment of Chondroitin Sulfate Particles Containing rhBMP-2 and BSA Particles Containing Vancomycin

In order to modify negatively charged particles due to the presence of chondroitin sulfate formed on the surface of the chondroitin sulfate particles containing rhBMP-2 to positively charged particles, 500 μl of 0.05 mg/ml polyethyleneimine (PEI) aqueous solution was added to the chondroitin sulfate particles containing rhBMP-2 prepared in Example 5-2 and gently stirred for 3 hours. Then, the resulting particles were collected by centrifugation at 13,000 rpm for 30 minutes, washed with distilled water three times to remove unreacted PEI, and freeze-dried at −20° C., thus preparing chondroitin sulfate particles containing rhBMP-2, coated with positively charged PEI.

Moreover, in order to modify the surface of the BSA particles containing vancomycin to a positively charged surface, 1 ml of 0.05 wt % poly-L-lysine (PLL) was added to the BSA particles containing vancomycin prepared in Example 5-2 and stirred for 4 hours. Then, the resulting particles were collected by centrifugation, washed with distilled water three times, and freeze-dried, thus preparing BSA particles containing vancomycin, coated with positively charged PLL.

5-4. Immobilization of Chondroitin Sulfate Particles Containing rhBMP-2 and BSA Particles Containing Vancomycin on Titanium Surface

1 mg of the chondroitin sulfate particles containing rhBMP-2, coated with PEI, a positively charged polymer, and 5 mg of the BSA particles containing vancomycin, coated with PLL, a positively charged polymer, which were prepared in Example 5-3 were dispersed in distilled water. Then, the titanium discs prepared in Example 5-1 were placed in the distilled water in which the chondroitin sulfate particles and the BSA particles were dispersed, and slowly stirred for 4 hours. Subsequently, the resulting titanium discs were washed with distilled water at least three times to remove an excess of particles, which were not immobilized on the surface, and freeze-dried at −20° C., thus preparing the titanium on which the chondroitin sulfate particles containing rhBMP-2 and the BSA particles containing vancomycin on titanium surface were immobilized.

It was observed that the average amount of the prepared chondroitin sulfate particles containing rhBMP-2 immobilized on the titanium surface was 1.9 μg and the average amount of the prepared BSA particles containing vancomycin immobilized on the titanium surface was 2.8 μg. Moreover, it was found that the chondroitin sulfate particles containing rhBMP-2 and the BSA particles containing vancomycin were well dispersed and immobilized on the titanium surface.

### Experimental Example 1

### Measurement of Change in Release Behavior of Therapeutic Agent

One (n=6) of the titanium discs on which the PLGA particles containing the rhBMP-2 were immobilized and one (n=6) of the apatite-coated titanium discs on which the PLGA particles containing the rhBMP-2 were immobilized, which are prepared in Example 4, were placed in 2 ml of phosphate buffer solution (PBS, pH 7.4) and then placed in a incubator at 37° C. for 30 days, and a predetermined amount of PBS was collected to analyze the amount of rhBMP-2 released from the titanium discs. The amount of rhBMP-2 released from the titanium discs was measured using an enzyme-linked immunosorbent assay (ELISA) kit (Pepro Tech, USA) (refer to Peprotech Human BMP-2 ELISA Development Kit 900-K225; http://www.peprotech.com/pdf/900-K225%20Human %20BMP-2%20EDK%20Lot%200305255.pdf).

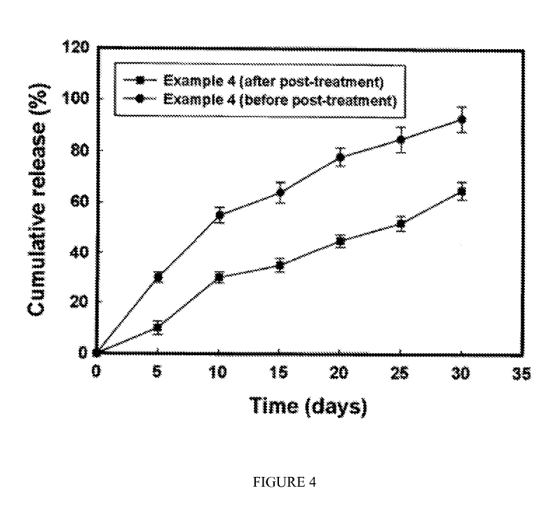

The results are shown in FIG. 4.

As shown in FIG. 4, in the case of the titanium disc which was not subjected to the post-treatment by apatite coating, the amount of rhBMP-2, a growth factor, released was rapidly increased and more than 90% percent was released on the 30th day, while in the case of the titanium disc which was subjected to the post-treatment by apatite coating, the release rhBMP-2 occurred slowly and only about 50% was released even on the 30th day, from which it can be seen that the release of the growth factor occurs stably and continuously. That is, with the apatite coating performed as the post-treatment after the immobilization of the particles, it is possible to improve the adhesion of the particles with respect to the metal alloy surface and also to substantially prevent the loss of the therapeutic agent, and it can be seen that it is possible to effectively prepare the metal alloy material suitable for the therapeutic agent with a long-term release.

As described above, according to the biomedical metal alloy material of the present invention, on which the particles containing the therapeutic agent are immobilized, it is possible to simply and effectively incorporate a plurality of therapeutic agent of different types on the surface of the metal alloy material, compared to conventional methods, to control the diameter of the particles containing the therapeutic agent from several tens of nanometers to several hundreds of micrometers, to use any therapeutic agent having a charge, regardless of the raw material, shape, strength, porosity, surface function, etc. of the metal alloy material, and to effectively control the number, content, and release rate of therapeutic agents to be introduced into the metal alloy material, and thus it is possible to effectively improve the biocompatibility, treatment effect, inflammatory reaction, foreign body reaction, stem cell differentiation inducing efficiency, etc. and, at the same time, to significantly improve the tissue regeneration and healing of implantation sites, the cell affinity to implantation sites, and the inflammatory reaction by inducing active tissue regeneration.

It will be apparent to those skilled in the art that various modifications can be made to the above-described exemplary embodiments of the present invention without departing from the spirit or scope of the invention. Thus, it is intended that the present invention covers all such modifications provided they come within the scope of the appended claims and their equivalents.

What is claimed is:

1. A method for preparing a biomedical metal alloy material with a multi-drug delivery system, comprising the steps of:
(1) incorporating a therapeutic agent into a biodegradable material to prepare particles containing the therapeutic agent;
(2) coating the surface of the particles containing the therapeutic agent with an ionic polymer having a positive charge or a negative charge to treat the surface of the particles containing the therapeutic agent to have a charge opposite to the surface charge of a metal alloy material; and
(3) dispersing the surface-treated particles containing the therapeutic agent in a solvent, adding the metal alloy material thereto, and stirring the resulting mixture to induce an electrostatic interaction between the surface charges of the particles containing the therapeutic agent and the metal alloy material to immobilize the particles containing the therapeutic agent on the surface of the metal alloy material.

2. The method of claim 1, wherein the biodegradable material comprises at least one selected from the group consisting of polydioxanone, polyglycolic acid, polylactic acid, polycaprolactone, lactic acid-glycolic acid copolymer, glycolic acid-trimethylcarbonate, glycolic acid-ε-caprolactone, polyglyconate, polyglactin, polyamino acid, polyanhydride, polyorthoester, and mixtures and copolymers thereof; collagen, gelatine, chitin/chitosan, alginate, albumin, hyaluronic acid, heparin, fibrinogen, cellulose, dextran, pectin, polylysine, polyethyleneimine, dexamethasone, chondroitin sulfate, lysozyme, DNA, RNA, Arg-Gly-Asp (RGD); and lipid, growth factors, growth hormones, peptide drugs, protein drugs, anti-inflammatory analgesic agents, anticancer agents, antiviral agents, sex hormones, antibiotics, antimicrobial agents, and mixtures thereof.

3. The method of claim 1, wherein the therapeutic agent comprises at least one selected from the group consisting of growth factors, growth hormones, peptide drugs, protein drugs, anti-inflammatory analgesic agents, anticancer agents, antiviral agents, sex hormones, antibiotics, antimicrobial agents, and mixtures thereof.

4. The method of claim 3, wherein the therapeutic agent comprises at least one selected from the group consisting of transforming growth factors, fibroblast growth factors, bone morphogenetic proteins, vascular endothelial growth factors, epidermal growth factors, insulin-like growth factors, platelet-derived growth factors, nerve growth factors, hepatocyte growth factors, placental growth factors, granulocyte colony-stimulating factors, animal growth hormones, human growth hormones, chondroitin sulfate, heparin, erythropoietin, granulocyte colony-stimulating factors, interferon, follicle-stimulating hormones, luteinizing hormones, goserelin acetate, leuprorelin acetate, decapeptyl, luteinizing hormone-releasing hormone agonists, dexamethasone, indomethacin, ibuprofen, ketoprofen, piroxicam, flurbiprofen, diclofenac, paclitaxel, doxorubicin, camptothecin, 5-fluorouracil, cytosine arabinose, methotrexate, acyclovir, robavin, tamiflu, testosterone, estrogen, progesterone, estradiol, tetracycline, minocycline, doxycycline, ofloxacin, levofloxacin, ciprofloxacin, clarithromycin, erythromycin, cefaclor, cefotaxime, imipenem, penicillin, gentamicin, streptomycin, vancomycin, ketoconazole, itraconazole, fluconazole, amphotericin-B, nystatin, griseofulvin, β-glycerophosphate, ascorbate, hydrocortisone, and 5-azacytidine.

5. The method of claim 1, wherein the particles containing the therapeutic agent prepared in step (1) is prepared by at least one method selected from the group consisting of a water-in-oil emulsion method, water-in-oil-in-water emulsion method, a spraying method, a solvent diffusion method, a phase separation method, a method for forming an intermolecular electrolyte complex between materials with charges, and a liposome method.

6. The method of claim 1, wherein the particles containing the therapeutic agent prepared in step (1) is non-porous or has a porosity of 5% to 98%.

7. The method of claim 1, wherein the particles containing the therapeutic agent prepared in step (1) has a diameter of 10 nm to 500 μm.

8. The method of claim 1, further comprising, before steps (2) and (3) the step of pretreating the particles containing the therapeutic agent and the metal alloy material by plasma treatment.

9. The method of claim 8, wherein the plasma treatment is performed in a non-vacuum environment or in a vacuum of less than 200 mtorr in the presence of at least one gas selected from the group consisting of oxygen, argon, hydrogen peroxide, and ammonia at 10 to 200 Watt for 1 to 5 minutes.

10. The method of claim 1, wherein in step (2), the ionic polymer having a positive charge or a negative charge comprises at least one cationic polymer selected from the group consisting of polyethyleneimine, polylysine, polyallylamine, polyvinylamine, poly(diallyldimethylammonium chloride), poly methyl aminoethyl methacrylate, N-hydroxysuccinimide, N-3-dimethylaminopropyl-N′-ethylcarbodiimide hydrochloride, chitosan, lysozyme, dextran, protein, and vancomycin or at least one anionic polymer selected from the group consisting of polydioxanone, polyglycolic acid, polylactic acid, polycaprolactone, lactic acid-glycolic acid copolymer, glycolic acid-trimethylcarbonate, glycolic acid-ε-caprolactone, polyglyconate, polyglactin, and copolymers thereof, collagen, heparin, albumin, hyaluronic acid, chondroitin sulfate, hydrochloride carboxymethylcellulose, sodium tripolyphosphate, polystyrene sulfonate, gelatin, and alginate.

11. The method of claim 1, wherein the metal alloy material comprises at least one selected from the group consisting of iron, chrome, nickel, stainless steel, cobalt-based alloy, titanium, titanium alloy, zirconium, niobium, tantalum, gold, and silver.

12. The method of claim 1, wherein the metal alloy material comprises at least one form selected from the group consisting of block, film, filament, fiber, membrane, mesh, woven fabric/non-woven fabric, knit, grains, particles, plate, bolt/nut, and nail.

13. The method of claim 1, wherein the metal alloy material is non-porous or has a porosity of 5% to 98%.

14. The method of claim 1, wherein the metal alloy material has a pore size of 0.1 nm to 5 mm.

15. The method of claim 1, wherein step (3) further comprises the step of pretreating the metal alloy material by at least one method selected from the group consisting of plasma treatment, sintering of metal beads, blasting and acid treatment, alkali immersion and heat treatment, ceramic coating, anodic oxidation, ion implantation, and combinations thereof.

16. The method of claim 15, wherein the ceramic coating uses at least one material selected from the group consisting of hydroxyapatite (HA), tricalcium phosphate (TCP), tetracalcium phosphate (TTCP), dicalcium phosphate anhydrous (DCPA), silica-based glasses, phosphate-based glasses, glass ceramics, alumina, zirconia, and complexes thereof.

17. The method of claim 16, wherein the ceramic coating is performed by at least one method selected from the group consisting of ion beam sputtering, radio-frequency sputtering, pulsed laser deposition, plasma spray, super high speed blast coating, and simulated body fluid method.

18. The method of claim 1, wherein in step (3), the solvent comprises at least one selected from the group consisting of water, ethanol, methanol, acetone, heptane, pentane, and mixtures thereof.

19. The method of claim 1, wherein the particles containing the therapeutic agent immobilized in step (3) are in an amount of 10⁻⁷ wt % to 90 wt % with respect to the total weight of the metal alloy material.

20. The method of claim 1, further comprising, after step (3), the step of performing post-treatment by at least one step selected from the group consisting of (a) immersing the metal alloy material, on which the particles containing the therapeutic agent are immobilized, in a solvent and drying the resulting metal alloy material, (b) partially melting the metal alloy material on which the particles containing the therapeutic agent are immobilized, and (c) coating the surface of the metal alloy material, on which the particles containing the therapeutic agent are immobilized, with apatite.

21. The method of claim 20, wherein in step (a), the solvent comprises at least one selected from the group consisting of water, hydrochloric acid, acetic acid, ethanol, acetone, methanol, dichloromethane, chloroform, toluene, acetonitrile, 1,4-dioxane, tetrahydrofuran, hexafluoroisopropanol, and mixtures thereof.

22. The method of claim 20, wherein in step (b), the melting is performed at 30° C. to 300° C. for 10 seconds to 1 hour.

23. The method of claim 20, wherein in step (c), the coating with apatite is performed by an alternating immersion process of (i) immersing the metal alloy material, on which the particles containing the therapeutic agent are immobilized, in a 30% (v/v) to 90% (v/v) ethanol aqueous solution, in which 0.1 M to 1 M calcium chloride (CaCl₂) is dissolved, for 3 to 10 seconds, immersing the resulting metal alloy material in a pure 30% (v/v) to 90% (v/v) ethanol aqueous solution for 1 to 3 seconds, and drying the resulting metal alloy material at room temperature for 3 to 5 minutes and (ii) immersing the resulting metal alloy material in a 30% (v/v) to 90% (v/v) ethanol aqueous solution, in which 0.1 M to 1 M dipotassium phosphate (K₂HPO₄) is dissolved, for 3 to 10 seconds, immersing the resulting metal alloy material in a pure 30% (v/v) to 90% (v/v) ethanol aqueous solution for 1 to 3 seconds, and drying the resulting metal alloy material at room temperature for 3 to 5 minutes, the alternating immersion process being performed one to five times, and then by immersing the resulting metal alloy material, on which the particles containing the therapeutic agent are immobilized, in a simulated body fluid (SBF) solution at a temperature of 37±0.5° C. at a pH of 6.4 to 7.4 for 1 hour to 5 days.

24. A biomedical metal alloy material prepared by the method of claim 1 and comprising immobilized particles containing a therapeutic agent in an amount of 10⁻⁷ wt % to 90 wt % with respect to the total weight of the metal alloy material.
